(19) United States
(12) Patent Application Publication
(10) Pub. No.: US 2026/0165945 A1
(43) Pub. Date: Jun. 18, 2026

(54) NATURAL FORMULATIONS FOR HAIR CARE

(71) Applicant: Rejuva Kare Inc., Fairfield, CA (US)

(72) Inventor: Tondenisha Coleman, Fairfield, CA (US)

(21) Appl. No.: 19/416,100

(22) Filed: Dec. 11, 2025

Related U.S. Application Data
(60) Provisional application No. 63/733,215, filed on Dec. 12, 2024.

(51) Int. Cl. A61K 8/9789; A61K 8/35; A61K 8/60; A61K 8/67; A61K 8/92; A61K 8/9794; A61K 8/99; A61Q 5/02; A61Q 5/12

(52) U.S. Cl. CPC A61K 8/9789; A61K 8/35; A61K 8/602; A61K 8/678; A61K 8/922; A61K 8/9794; A61K 8/99; A61Q 5/02; A61Q 5/12; A61K 2800/522

(57) ABSTRACT

Disclosed in certain embodiments herein is a formulation for shampooing and conditioning the hair using natural components.

### FIELD OF THE INVENTION

The present invention relates to a formulation for hair care including natural components, which may be used to treat a variety of scalp conditions.

### BACKGROUND OF THE INVENTION

Currently, the hair care formulations used in the cosmetic industry containing a variety of synthetic components. There have been studies which have found that long term use of these components may enter the body and cause negative health effects. Therefore, there is a need to provide a formulation including mostly naturally components which can also achieve similar or better results when used.

### SUMMARY

It is an object of certain embodiments of the disclosure to provide a formulation for treating a variety of scalp conditions such as dryness or hair loss. It has been found that combining at least one moisturizer, at least one antioxidant, at least one preservative, and at least one antibacterial from natural ingredients was able to provide effective treatment to the scalp.

In an embodiment of the present disclosure, a formulation is provided. The formulation may include at least one moisturizer including argan oil, aloe vera, or a combination thereof; at least one antioxidant including chamomile; at least one preservative; and at least one antibacterial agent including camphor.

In some embodiments, the at least one moisturizer may further include sweet almond oil, castor oil, peppermint essential oil, or a combination thereof.

In some embodiments, the at least one preservative may include rosemary oleoresin, radish root ferment, Coco nucifera fruit with Lactobacillus, or a combination thereof.

In some embodiments, the at least one antibacterial agent may further include carrot oil, cypress essential oil, tea tree essential oil, or a combination thereof.

In some embodiments, the formulation may further include a humectant. In some embodiments, the humectant may include panthenol, honey glycerin, or a combination thereof.

In some embodiments, the formulation may further include a surfactant. In some embodiments, the surfactant may be a reactant of glucose and coconut oil.

In some embodiments, the formulation may formulation may further include a botanical agent. In some embodiments, the botanical agent may include rosemary essential oil, ylang ylang, hemp seed oil, lavender, eucalyptus, or a combination thereof.

In some embodiments, the formulation may further include an emollient. In some embodiments, the emollient may include shea butter, cocoa butter, mineral oil, lanolin, petrolatum, paraffin, beeswax, squalene, cetyl alcohol, stearyl alcohol, olive oil, triethylhexanoin, coconut oil, jojoba oil, sesame oil, almond oil, or combinations thereof.

In some embodiments, the formulation may further include water.

In some embodiments, the formulation may further include a natural gum. In some embodiments, the natural gum may include xanthan gum.

In another embodiment, an additional formulation is provided. The additional formulation may include at least one moisturizer comprising aloe vera; at least one emollient comprising coconut oil and sesame oil; at least one antibacterial agent comprising tea tree essential oil; and at least one antioxidant comprising Vitamin E.

In some embodiments, the at least one moisturizer may further include babassu oil, argan oil, sweet almond oil, castor oil, or peppermint essential oil.

In some embodiments, the at least one antibacterial agent may further include carrot oil, or cypress essential oil.

In some embodiments, the at least one antioxidant may further include dandelion extract.

In some embodiments, the additional formulation may further include a humectant. In some embodiments, the humectant may include panthenol, honey glycerin, or a combination thereof.

In some embodiments, the additional formulation may further include a surfactant. In some embodiments, the surfactant may be a reactant of glucose and coconut oil.

In some embodiments, the additional formulation may further include a botanical agent. In some embodiments, the botanical agent may include rosemary essential oil, ylang ylang, bilberry leaf extract, lavender, eucalyptus, or a combination thereof.

In some embodiment, the at least one emollient may further include shea butter, cocoa butter, mineral oil, lanolin, petrolatum, paraffin, beeswax, squalene, cetyl alcohol, stearyl alcohol, olive oil, triethylhexanoin, jojoba oil, almond oil, or combinations thereof.

In some embodiments, the additional formulation may further include water.

In some embodiments, the additional formulation may further include a natural gum. In some embodiments, the natural gum may include xanthan gum.

In some embodiments, the additional formulation may further include a quaternary ammonium salt. In some embodiments, the quaternary ammonium salt comprises behentrimonium methosulfate (BTMS).

### DETAILED DESCRIPTION

All percentages, parts and ratios used herein are by weight of the total composition and all measurements made are at room temperature, unless otherwise specified. All percentages, parts, and ratios are based upon the total weight of the compositions disclosed herein, unless otherwise specified.

As used herein, the terms “treatment” or “treating” with respect to a scalp condition generally mean “having positive effect on a scalp condition” and encompass reduction, amelioration, and/or alleviation of at least one symptom of a scalp condition, a reduction, amelioration, and/or alleviation in the severity of the scalp conditions, or delay, prevention, or inhibition of the progression of the scalp condition. Treatment, as used herein, therefore does not require total curing of the condition. A formulation of the present disclosure that is useful for treatment of a scalp condition, or a method of treating a scalp condition, need only reduce the severity of a scalp condition, reduce the severity of symptoms associated therewith, provide improvement to a patient's quality of life, or delay, prevent, or inhibit the onset of one or more symptoms of a scalp condition. As used herein, these terms also encompass aesthetic improvements to the scalp upon application of the disclosed formulations including improving moisturizing and hair loss.

As used herein, the terms “application,” “apply,” and “applying” with respect to a disclosed topical formulation or method of using a disclosed formulation, refer to any manner of administering a formulation to the scalp and/or hair. Smearing, rubbing, spreading, a disclosed formulation, with or without the aid of suitable devices, on a subject's scalp and/or hair are all included within the scope of the term “application,” as used herein.

As used herein, the phrase “effective amount” refers to an amount of a formulation of the present disclosure, or component thereof, effective to treat a scalp or hair condition as noted above, including a range of effects, from a detectable local improvement in an area of application to substantial relief of symptoms to an improvement in one or more aesthetic criteria, including, but not limited to, a perceived improvement in apparent scalp dryness, loss of hair, redness, or dryness. The effective amount will vary with the particular condition or conditions being treated, the severity of the condition, the duration of the treatment, the specific components of the composition being used, and other factors. The disclosed compositions, formulations and methods of use thereof reduce, minimize, or eliminate normally-observed dry scalp conditions including, inter alia, conditions characterized by keratosis pilaris, itching, severe skin flaking, breakdown of the skin barrier, discomfort, extreme dryness, cracking of the skin and sensitization. The disclosed compositions, formulations, and methods of use thereof also provide aesthetic improvements in the scalp and hair such as improve hair loss, and shininess.

### Shampoo Formulation

According to various embodiments, the present disclosure relates to a shampoo formulation including at least one moisturizer including argan oil or aloe vera; at least one antioxidant including chamomile; at least one preservative; and at least one antibacterial agent including camphor.

In some embodiments of the formulation, the at least one moisturizer may further include sweet almond oil, castor oil, peppermint essential oil, or a combination thereof.

In some embodiments, the at least one preservative may include rosemary oleoresin, radish root ferment, Cocos nucifera fruit with Lactobacillus, or a combination thereof.

In some embodiments, the at least one antibacterial agent may further include carrot oil, cypress essential oil, tea tree essential oil, or a combination thereof.

In some embodiments of the formulation, the at least one antioxidant may further include polyphenols, ascorbates, tetrahexyldecyl ascorbate, ubiquinone, green tea phenols, cinnamic acid, ferulic acid, caffeic acid, p-coumaric acid, sinapinic acid, cis and trans isomers thereof, salts thereof, equivalent derivatives thereof, and combinations thereof. In some embodiments, the antioxidant may be gallic acid, delphinidin, luteolin, quercetin, cyanidin, taxifolin, kaempferol, malvidin, hesperidin, pelargonidin, apigenin, naringenin, chrysin, ergothioneine, glutathione, emblica, cis and trans isomers thereof, salts thereof, equivalent derivatives thereof, and combinations thereof. In certain embodiments, the antioxidant may be apigenin, ergothioneine, glutathione, emblica, cis and trans isomers thereof, salts thereof, equivalent derivatives thereof, and combinations thereof. In some embodiments, the antioxidant may be anserine, ascorbic acid, butylated hydroxyanisole (BHA), butylated hydroxytoluene (BHT), cysteine, diosmine, hydroquinone, kojic acid, palmatine, pectolinarin, tannic acid, ubiquinone, cis and trans isomers thereof, salts thereof, equivalent derivatives thereof, and combinations thereof.

In some embodiments, the topical formulation may further include a humectant. Humectants are hygroscopic substances used to keep things moist, often a molecule with several hydrophilic groups, or capable of hydrogen bonding with water, or having other polar organic functional groups. Humectants can also function as solvents or cosolvents.

Some examples of humectants include, but are not limited to, amino acids; glycerin; glycols and polyols, such as propylene glycol, hexylene glycol, and butylene glycol, including polymeric and sugar-based polyols/alcohols, for example, glycerol, sorbitol, xylitol, maltitol, polydextrose; mucopolysaccharides and carbohydrates, for example, aloe vera gel, yucca extract, dextrose and polydextrose; alpha hydroxy acids such as glycolic acid, lactic acid; albumen; esters or amides of acetic acid or similar; soluble carbohydrates, such as sugar, honey; salts, such as potassium chloride, sodium PCA, salts of polycarboxylic acids; amides, such as urea and urea derivatives.

In some embodiments, the humectant may include panthenol, honey, glycerin, or a combination thereof.

In some embodiments, the formulation may further include a botanical agent. The botanical agent may be at least one of a rosemary essential oil, ylang ylang, hemp seed oil, lavender, eucalyptus, or a combination thereof. In other embodiments of the formulation, the botanical agent may be an aloe extract, an oat extract, a horse chestnut extract, an algae extract, a beet extract, an orange extract, a grapefruit extract, a lemon extract, a coconut extract, or a dandelion extract.

In certain embodiments, the formulation further includes a cosmetically acceptable excipient. Exemplary cosmetically acceptable excipients, include, without limitations, pH stabilizer, emollient, epidermal penetration enhancer, solvent, a surfactant, oil bodies, emulsifiers, pearlescent waxes, consistency regulators, thickeners, rheology modifiers, suspending agents, chelating agent, super fatting agents, stabilizers, fats, waxes, lecithins, phospholipids, Biogenic active ingredients, additional antioxidants, solubilizers, perfume oils, or natural or synthetic Triglycerides Including Glyceryl Esters and Derivatives.

In some embodiments, the moisturizer may be an aloe vera compound, argan oil, sweet almond oil, castor oil, peppermint essential oil, or a combination thereof. The aloe vera compound may be an aloe vera powder or an aloe vera extract.

Suitable solvents that may be used in the formulation described herein include, without limitations, water, alkanediols (e.g., ethylene glycol, propylene glycol, butylene glycol), ethoxylated or propoxylated diglycol, ethanol, propanol, isopropanol, glycerin, methoxyisopropanol, PPG-2 methyl ether, PPG-3 methyl ether, propylene glycol butyl ether, PPG-2 butyl ether, phenoxyisopropanol, butoxyethanol, butoxydiglycol, methoxydiglycol, phenoxyethanol, PPG-3 butyl ether, PPG-2 propyl ether, propylene glycol propyl ether, or dipropylene glycol dimethyl ether, or mixtures and combinations or individual ethoxylates, propoxylates and glyceryl esters thereof. In some embodiments, the solvent may be water.

In certain embodiments, the cosmetically acceptable excipient includes natural gums (e.g., a natural plant gum). Suitable natural gums include, without limitations, guar gum, carob gum, konjac gum, xanthan gum, sclerotium gum, acacia gum, cellulose gum (modified or not), or a combination thereof. In one embodiment, the natural gum in the formulation is xanthan gum.

In certain embodiments, the cosmetically acceptable excipient includes additional antioxidants such as a form of Vitamin E. Suitable forms of Vitamin E that may be included in the formulation can be selected from alpha, beta, delta, and gamma tocopherols, and alpha, beta, delta and gamma tocotrienols, and combinations thereof.

In certain embodiments, the cosmetically acceptable excipient in the formulation includes a preservative. Suitable preservative agents include, for example, phenoxyethanol, a solution of paraben, pentanediol and sorbic acid, as well as silver complexes which are known under the commercial reference Surfacine® and other classes of substances set out in annex 6, parts A and B of the cosmetic regulations, i.e. a suitable preservative, rosemary oleoresin, radish root ferment, Cocos nucifera fruit with Lactobacillus, or a combination thereof.

In certain embodiments, the cosmetically acceptable excipient in the formulation includes an emollient. The emollient may include shea butter, cocoa butter, mineral oil, lanolin, petrolatum, paraffin, beeswax, squalene, cetyl alcohol, stearyl alcohol, olive oil, triethylhexanoin, coconut oil, jojoba oil, sesame oil, almond oil, or combinations thereof.

In certain embodiments, the formulation may further include a surfactant. The surfactant may be an anionic surfactant, a non-ionic surfactant, or a combination thereof. In some embodiments, the non-ionic surfactant may include an ethoxylate, a fatty ethoxylate, a fatty polyglycoside, a propoxylate, a block copolymer, a polyglyceryl ester, a polyglycerol emulsifier, a sorbitol anhydride, a dipolar agent, a phenolic non-ionic, or a combination thereof. In another embodiment, the non-ionic surfactant may include decyl glucoside. In some embodiments, the surfactant may include a reactant of glucose and coconut oil.

In certain embodiments, the cosmetically acceptable excipient includes a perfume oil. Suitable perfume oils include mixtures of natural and synthetic fragrances. Natural fragrances are extracts from flowers (lily, lavender, rose, jasmine, neroli, ylang-ylang), stems and leaves (geranium, patchouli, petitgrain), fruits (aniseed, coriander, cumin, juniper), fruit peels (bergamot, lemon, orange), roots (mace, angelica, celery, cardamom, costus, iris, calmus), woods (pinewood, sandalwood, guaiac wood, cedarwood, rosewood), herbs and grasses (tarragon, lemongrass, sage, thyme), needles and branches (spruce, fir, pine, dwarf-pine), resins and balsams (galbanum, elemi, benzoin, myrrh, olibanum, opoponax).

Essential oils of relatively low volatility, which are mostly used as aroma components, are also suitable as perfume oils, e.g. sage oil, chamomile oil, oil of cloves, melissa oil, mint oil, cinnamon leaf oil, linden blossom oil, juniper berry oil, vetiver oil, olibanum oil, galbanum oil, labolanum oil and lavandin oil. Other suitable oils include bergamot oil, dihydromyrcenol, lilial, lyral, citronellol, phenylethyl alcohol, α-hexylcinnamaldehyde, geraniol, benzylacetone, cyclamen aldehyde, linalool, boisambrene forte, ambroxan, indole, hedione, sandelice, lemon oil, mandarin oil, orange oil, allyl amyl glycolate, cyclovertal, lavandin oil, clary sage oil, β-damascone, geranium oil bourbon, cyclohexyl salicylate, Vertofix coeur, iso-E-super, Fixolide NP, evernyl, iraldein gamma, phenylacetic acid, geranyl acetate, benzyl acetate, rose oxide, romilat, irotyl and floramat alone or in mixtures.

In certain embodiments, the cosmetically acceptable excipient includes a perfume oil that is an essential oil selected from the group of a lavender oil, a bergamot oil, a eucalyptus oil, a chamomile oil, a melaleuca oil, or a combination thereof. In one embodiment, the cosmetically acceptable excipient includes a lavender oil, a chamomile oil, or a combination thereof.

In some embodiments, the formulations described herein may be formulated as a shampoo.

The formulations according to the disclosure may also contain one or one more additional cosmetically acceptable excipients as described below.

### Natural or Synthetic Triglycerides Including Glyceryl Esters and Derivatives

Di- or triglycerides, based on C₆-C₁₈ fatty acids, modified by reaction with other alcohols (caprylic/capric triglyceride, wheat germ glycerides, etc.). Fatty acid esters of polyglycerin (polyglyceryl-n such as polyglyceryl-4 caprate, polyglyceryl-2 isostearate, etc. or castor oil, hydrogenated vegetable oil, sweet almond oil, wheat germ oil, sesame oil, hydrogenated cottonseed oil, coconut oil, avocado oil, corn oil, hydrogenated castor oil, shea butter, cocoa butter, soybean oil, mink oil, sunflower oil, safflower oil, macadamia nut oil, olive oil, hydrogenated tallow, apricot kernel oil, hazelnut oil, borage oil, etc.

Waxes including esters of long-chain acids and alcohols as well as compounds having wax-like properties, e.g., carnauba wax, beeswax (white or yellow), lanolin wax, candelilla wax, ozokerite, japan wax, paraffin wax, microcrystalline wax, ceresin, cetearyl esters wax, synthetic beeswax, etc. Also, hydrophilic waxes as Cetearyl Alcohol or partial glycerides.

### Surfactants

Examples of suitable mild surfactants, that is to say surfactants especially well tolerated by the skin, include fatty alcohol polyglycol ether sulfates, monoglyceride sulfates, mono- and/or di-alkyl sulfosuccinates, fatty acid isethionates, fatty acid sarcosinates, fatty acid taurides, fatty acid glutamates, .alpha.-olefin sulfonates, ethercarboxylic acids, alkyl oligoglucosides, fatty acid glucamides, alkylamidobetaines and/or protein fatty acid condensation products, the latter preferably being based on wheat proteins.

### Biogenic Active Ingredients

Biogenic active ingredients are to be understood as meaning, for example, tocopherol, tocopherol acetate, tocopherol palmitate, deoxyribonucleic acid, retinol, bisabolol, allantoin, phytantriol, AHA acids, amino acids, ceramides, pseudoceramides, essential oils, plant extracts and vitamin complexes.

### Antioxidants

Typical examples of such antioxidants are amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotinoids, carotenes, lycopene and derivatives thereof, chlorogenic acid and derivatives thereof, lipoic acid and derivatives thereof (e.g. dihydrolipoic acid), aurothioglycose, propylthiouracil and other thiols (e.g. thioredoxin, glutathione, cysteine, cystine, cystamine and the glycosyl, N-acetyl, methyl, ethyl, propyl, amyl, butyl, lauryl, palmitoyl, oleyl, linoleyl, cholesteryl and glyceryl esters thereof) and also salts thereof, dilauryl thiodipropionate, distearyl thiodipropionate, thiodipropionic acid and derivatives thereof (esters, ethers, peptides, lipids, nucleotides, nucleosides and salts) and also sulfoximine compounds (e.g. buthionine sulfoximines, homocysteine sulfoximine, buthionine sulfones, penta-, hexa-, hepta-thionine sulfoximine), also (metal) chelating agents (e.g. hydroxy fatty acids, palmitic acid phytic acid, lactoferrin), hydroxy acids (e.g. citric acid, lactic acid, malic acid), humic acid, bile acid, bile extracts, bilirubin, biliverdin, EDTA, EDDS, EGTA and derivatives thereof, unsaturated fatty acids and derivatives thereof (e.g. linolenic acid, linoleic acid, oleic acid), folic acid and derivatives thereof, ubiquinone and ubiquinol and derivatives thereof, vitamin C and derivatives (e.g. ascorbyl palmitate, magnesium ascorbyl phosphate, ascorbyl acetate), tocopherols and derivatives (e.g. vitamin E acetate), vitamin A and derivatives (e.g. vitamin A palmitate) and also coniferyl benzoate of benzoin resin, rutinic acid and derivatives thereof, glycosylrutin, ferulic acid, furfurylidene glucitol, carnosine, butyl hydroxytoluene, butyl hydroxyanisole, nordihydroguaiaretic acid, trihydroxybutyrophenone, uric acid and derivatives thereof, mannose and derivatives thereof, superoxide dismutase, N-[3-(3,5-di-tert-butyl-4-hydroxyphenyl) propionyl]sulfanilic acid (and salts thereof, for example the disodium salts), selenium and derivatives thereof (e.g. selenium methionine), stilbene and derivatives thereof (e.g. stilbene oxide, trans-stilbene oxide) and the derivatives suitable according to the invention (salts, esters, ethers, sugars, nucleotides, nucleosides, peptides and lipids) of those mentioned active ingredients. HALS (=“Hindered Amine Light Stabilizers”) compounds may also be mentioned.

### Preservatives and Bacteria-Inhibiting Agents

Suitable preservatives include, for example, Methyl-, Ethyl-, Propyl-, Butyl-parabens, Benzalkonium chloride, 2-Bromo-2-nitro-propane-1,3-diol, Dehydroacetic acid, Diazolidinyl Urea, 2-Dichloro-benzyl alcohol, DMDM hydantoin, Formaldehyde solution, Methyldibromoglutanitrile, Phenoxyethanol, Sodium Hydroxymethylglycinate, Imidazolidinyl Urea, Triclosan and further substance classes listed in the following reference: K. F. DePolo-A short textbook of cosmetology, Chapter 7, Table 7-2, 7-3, 7-4 and 7-5, p 210-219.

In certain embodiments, the formulation may include at least one moisturizer; at least one antioxidant; at least one preservative; and at least one antibacterial agent.

In some embodiments, the at least one moisturizer may be included in an amount of about 0.01 wt % to about 5 wt %, about 0.05 wt % to about 4.5 wt %, about 0.1 wt % to about 4 wt %, about 0.2 wt % to about 3.5 wt %, about 0.5 wt % to about 3 wt %, or about 1 wt % to about 2.5 wt %, based on total weight of the formulation.

In some embodiments, the at least one antioxidant may be included in an amount of about 0.5 wt % to about 5 wt %, about 1 wt % to about 4.5 wt %, about 1.5 wt % to about 4 wt %, about 2 wt % to about 3.5 wt %, or about 2.5 wt % to about 3 wt %, based on total weight of the formulation.

In some embodiments, the at least one preservative may be included in an amount of about 1 wt % to about 35 wt %, about 5 wt % to about 30 wt %, about 10 wt % to about 25 wt %, or about 15 wt % to about 20 wt %, based on total weight of the formulation.

In some embodiments, the at least one antibacterial agent may be included in an amount of about 1 wt % to about 10 wt %, about 2 wt % to about 9 wt %, about 3 wt % to about 8 wt %, or about 4 wt % to about 6 wt %, based on total weight of the formulation.

In some embodiments, the humectant may be included in an amount of about 0.1 wt % to about 5 wt %, about 0.5 wt % to about 4.5 wt %, about 1 wt % to about 4 wt %, about 1.5 wt % to about 3.5 wt %, or about 2 wt % to about 3 wt %, based on total weight of the formulation.

In some embodiments, the surfactant may be included in an amount of about 10 wt % about 40 wt %, about 15 wt % to about 35 wt %, or about 20 wt % to about 30 wt %, based on total weight of the formulation.

In some embodiments, the at least one cosmetically acceptable excipient may be included in an amount of about 0.001 wt % to about 25 wt %, about 0.005 wt % to about 22 wt %, about 0.01 wt % to about 20 wt %, about 0.05 wt % to about 18 wt %, about 0.1 wt % to about 15 wt %, about 0.5 wt % to about 12 wt %, about 1 wt % to about 10 wt %, about 2 wt % to about 8 wt %, or about 3 wt % to about 5 wt %, based on total weight of the topical formulation.

In another embodiment of the formulation, the formulation may include argan oil, an aloe vera component, chamomile, camphor, sweet almond oil, castor oil, peppermint essential oil, rosemary oleoresin, radish root ferment, Cocos nucifera fruit with Lactobacillus, carrot oil, cypress essential oil, tea tree essential oil, panthenol, honey, glycerin, a reactant of glucose and coconut oil, rosemary essential oil, hemp see oil, ylang ylang, lavender, eucalyptus, coconut oil, jojoba oil, almond oil, sesame oil, xanthan gum, and water.

In some embodiments, the shampoo formulation may include jojoba oil to aid in thickening of hair follicles and prevent hair loos. In some embodiments, the shampoo formulation may include ylang ylang to help improve blood circulation. In some embodiments, camphor may be included to treat head lice and/or fungal infection and/or dandruff. In some embodiments, rosehip seed oil may reduce split ends, or prevent or treat dandruff. In some embodiments, echinacea may be included to help hair growth and prevent early onset hair loss. In some embodiments, chamomile may be included to relive inflammation. In some embodiments, lavender may be included as an antibiotic in the formulation. In some embodiments, eucalyptus may be included as an antibacterial agent.

In some embodiments, the formulation may be formulated as a shampoo.

In some embodiments, a method of applying the formulations are provided. The methods including applying the formulations described herein to the scalp and/or hair.

A method of preparing the formulation for shampooing is also provided. The method includes combining a humectant, natural gum, and at least one moisturizer and mixing until smooth. Once smooth, adding water to the mixture and mixing the same with a hand mixer. The method may further include combining the remaining ingredients except the surfactant with the mixture. Once combined, the surfactant is combined at high speed mixing until the mixture is smooth and homogeneous. The formulation may be prepared at room temperature.

### Additional Formulation for Conditioning

According to various embodiments, the present disclosure relates to a formulation for conditioning the hair including at least one moisturizer including aloe vera; at least one emollient including coconut oil and sesame oil; at least one antibacterial gent including tea tree essential oil; and at least one antioxidant including Vitamin E.

In some embodiments of the formulation, the at least one moisturizer may further include babassu oil, argan oil, sweet almond oil, castor oil, peppermint essential oil, or a combination thereof.

In some embodiments, the at least one antibacterial agent may further include carrot oil, cypress essential oil, or a combination thereof.

In some embodiments, the at least one antioxidant may further include dandelion extract.

In some embodiments, the formulation for conditioning may further include t least one preservative may include rosemary oleoresin, radish root ferment, Cocos nucifera fruit with Lactobacillus, or a combination thereof.

In some embodiments of the formulation, the at least one antioxidant may further include polyphenols, ascorbates, tetrahexyldecyl ascorbate, ubiquinone, green tea phenols, cinnamic acid, ferulic acid, caffeic acid, p-coumaric acid, sinapinic acid, cis and trans isomers thereof, salts thereof, equivalent derivatives thereof, and combinations thereof. In some embodiments, the antioxidant may be gallic acid, delphinidin, luteolin, quercetin, cyanidin, taxifolin, kaempferol, malvidin, hesperidin, pelargonidin, apigenin, naringenin, chrysin, ergothioneine, glutathione, emblica, cis and trans isomers thereof, salts thereof, equivalent derivatives thereof, and combinations thereof. In certain embodiments, the antioxidant may be apigenin, ergothioneine, glutathione, emblica, cis and trans isomers thereof, salts thereof, equivalent derivatives thereof, and combinations thereof. In some embodiments, the antioxidant may be anserine, ascorbic acid, butylated hydroxyanisole (BHA), butylated hydroxytoluene (BHT), cysteine, diosmine, hydroquinone, kojic acid, palmatine, pectolinarin, tannic acid, ubiquinone, cis and trans isomers thereof, salts thereof, equivalent derivatives thereof, and combinations thereof.

In some embodiments, the formulation may further include a humectant. Humectants are hygroscopic substances used to keep things moist, often a molecule with several hydrophilic groups, or capable of hydrogen bonding with water, or having other polar organic functional groups. Humectants can also function as solvents or cosolvents.

Some examples of humectants include, but are not limited to, amino acids; glycerin; glycols and polyols, such as propylene glycol, hexylene glycol, and butylene glycol, including polymeric and sugar-based polyols/alcohols, for example, glycerol, sorbitol, xylitol, maltitol, polydextrose; mucopolysaccharides and carbohydrates, for example, aloe vera gel, yucca extract, dextrose and polydextrose; alpha hydroxy acids such as glycolic acid, lactic acid; albumen; esters or amides of acetic acid or similar; soluble carbohydrates, such as sugar, honey; salts, such as potassium chloride, sodium PCA, salts of polycarboxylic acids; amides, such as urea and urea derivatives.

In some embodiments, the humectant may include panthenol, honey, glycerin, or a combination thereof.

In some embodiments, the formulation may further include a botanical agent. The botanical agent may be at least one of a rosemary essential oil, ylang ylang, bilberry leaf extract, lavender, eucalyptus, or a combination thereof. In other embodiments of the formulation, the botanical agent may be an aloe extract, an oat extract, a horse chestnut extract, an algae extract, a beet extract, an orange extract, a grapefruit extract, a lemon extract, a coconut extract, or a dandelion extract.

In certain embodiments, the formulation further includes a cosmetically acceptable excipient. Exemplary cosmetically acceptable excipients, include, without limitations, pH stabilizer, emollient, epidermal penetration enhancer, solvent, a surfactant, oil bodies, emulsifiers, pearlescent waxes, consistency regulators, thickeners, rheology modifiers, suspending agents, chelating agent, super fatting agents, stabilizers, fats, waxes, lecithins, phospholipids, Biogenic active ingredients, additional antioxidants, solubilizers, perfume oils, or natural or synthetic Triglycerides Including Glyceryl Esters and Derivatives.

In some embodiments, the moisturizer may be an aloe vera compound, argan oil, sweet almond oil, castor oil, peppermint essential oil, or a combination thereof. The aloe vera compound may be an aloe vera powder or an aloe vera extract.

Suitable solvents that may be used in the formulation described herein include, without limitations, water, alkanediols (e.g., ethylene glycol, propylene glycol, butylene glycol), ethoxylated or propoxylated diglycol, ethanol, propanol, isopropanol, glycerin, methoxyisopropanol, PPG-2 methyl ether, PPG-3 methyl ether, propylene glycol butyl ether, PPG-2 butyl ether, phenoxyisopropanol, butoxyethanol, butoxydiglycol, methoxydiglycol, phenoxyethanol, PPG-3 butyl ether, PPG-2 propyl ether, propylene glycol propyl ether, or dipropylene glycol dimethyl ether, or mixtures and combinations or individual ethoxylates, propoxylates and glyceryl esters thereof. In some embodiments, the solvent may be water.

In certain embodiments, the cosmetically acceptable excipient includes natural gums (e.g., a natural plant gum). Suitable natural gums include, without limitations, guar gum, carob gum, konjac gum, xanthan gum, sclerotium gum, acacia gum, cellulose gum (modified or not), or a combination thereof. In one embodiment, the natural gum in the formulation is xanthan gum.

In certain embodiments, suitable forms of Vitamin E that may be included in the formulation can be selected from alpha, beta, delta, and gamma tocopherols, and alpha, beta, delta and gamma tocotrienols, and combinations thereof.

In certain embodiments, the cosmetically acceptable excipient in the formulation includes a preservative. Suitable preservative agents include, for example, phenoxyethanol, a solution of paraben, pentanediol and sorbic acid, as well as silver complexes which are known under the commercial reference Surfacine® and other classes of substances set out in annex 6, parts A and B of the cosmetic regulations, i.e. a suitable preservative, rosemary oleoresin, radish root ferment, Cocos nucifera fruit with Lactobacillus, or a combination thereof.

In certain embodiments, the cosmetically acceptable excipient in the formulation includes an emollient. The emollient may include shea butter, cocoa butter, mineral oil, lanolin, petrolatum, paraffin, beeswax, squalene, cetyl alcohol, stearyl alcohol, olive oil, triethylhexanoin, coconut oil, jojoba oil, sesame oil, almond oil, or combinations thereof.

In certain embodiments, the formulation may further include a surfactant. The surfactant may be an anionic surfactant, a non-ionic surfactant, or a combination thereof. In some embodiments, the non-ionic surfactant may include an ethoxylate, a fatty ethoxylate, a fatty polyglycoside, a propoxylate, a block copolymer, a polyglyceryl ester, a polyglycerol emulsifier, a sorbitol anhydride, a dipolar agent, a phenolic non-ionic, or a combination thereof. In another embodiment, the non-ionic surfactant may include decyl glucoside. In some embodiments, the surfactant may include a reactant of glucose and coconut oil.

In certain embodiments, the cosmetically acceptable excipient includes a perfume oil. Suitable perfume oils include mixtures of natural and synthetic fragrances. Natural fragrances are extracts from flowers (lily, lavender, rose, jasmine, neroli, ylang-ylang), stems and leaves (geranium, patchouli, petitgrain), fruits (aniseed, coriander, cumin, juniper), fruit peels (bergamot, lemon, orange), roots (mace, angelica, celery, cardamom, costus, iris, calmus), woods (pinewood, sandalwood, guaiac wood, cedarwood, rosewood), herbs and grasses (tarragon, lemongrass, sage, thyme), needles and branches (spruce, fir, pine, dwarf-pine), resins and balsams (galbanum, elemi, benzoin, myrrh, olibanum, opoponax).

Essential oils of relatively low volatility, which are mostly used as aroma components, are also suitable as perfume oils, e.g. sage oil, chamomile oil, oil of cloves, melissa oil, mint oil, cinnamon leaf oil, linden blossom oil, juniper berry oil, vetiver oil, olibanum oil, galbanum oil, labolanum oil and lavandin oil. Other suitable oils include bergamot oil, dihydromyrcenol, lilial, lyral, citronellol, phenylethyl alcohol, α-hexylcinnamaldehyde, geraniol, benzylacetone, cyclamen aldehyde, linalool, boisambrene forte, ambroxan, indole, hedione, sandelice, lemon oil, mandarin oil, orange oil, allyl amyl glycolate, cyclovertal, lavandin oil, clary sage oil, β-damascone, geranium oil bourbon, cyclohexyl salicylate, Vertofix coeur, iso-E-super, Fixolide NP, evernyl, iraldein gamma, phenylacetic acid, geranyl acetate, benzyl acetate, rose oxide, romilat, irotyl and floramat alone or in mixtures.

In certain embodiments, the cosmetically acceptable excipient includes a perfume oil that is an essential oil selected from the group of a lavender oil, a bergamot oil, a eucalyptus oil, a chamomile oil, a melaleuca oil, or a combination thereof. In one embodiment, the cosmetically acceptable excipient includes a lavender oil, a chamomile oil, or a combination thereof.

In some embodiments, the formulation for conditioning may further include a quaternary ammonium salt. In some embodiments, the quaternary ammonium slat may include behentrimonium methosulfate (BTMS).

In some embodiments, the formulations described herein may be formulated as a conditioner.

The formulations according to the disclosure may also contain one or one more additional cosmetically acceptable excipients as described below.

### Natural or Synthetic Triglycerides Including Glyceryl Esters and Derivatives

Di- or triglycerides, based on C₆-C₁₈ fatty acids, modified by reaction with other alcohols (caprylic/capric triglyceride, wheat germ glycerides, etc.). Fatty acid esters of polyglycerin (polyglyceryl-n such as polyglyceryl-4 caprate, polyglyceryl-2 isostearate, etc. or castor oil, hydrogenated vegetable oil, sweet almond oil, wheat germ oil, sesame oil, hydrogenated cottonseed oil, coconut oil, avocado oil, corn oil, hydrogenated castor oil, shea butter, cocoa butter, soybean oil, mink oil, sunflower oil, safflower oil, macadamia nut oil, olive oil, hydrogenated tallow, apricot kernel oil, hazelnut oil, borage oil, etc.

Waxes including esters of long-chain acids and alcohols as well as compounds having wax-like properties, e.g., carnauba wax, beeswax (white or yellow), lanolin wax, candelilla wax, ozokerite, japan wax, paraffin wax, microcrystalline wax, ceresin, cetearyl esters wax, synthetic beeswax, etc. Also, hydrophilic waxes as Cetearyl Alcohol or partial glycerides.

### Surfactants

Examples of suitable mild surfactants, that is to say surfactants especially well tolerated by the skin, include fatty alcohol polyglycol ether sulfates, monoglyceride sulfates, mono- and/or di-alkyl sulfosuccinates, fatty acid isethionates, fatty acid sarcosinates, fatty acid taurides, fatty acid glutamates, .alpha.-olefin sulfonates, ethercarboxylic acids, alkyl oligoglucosides, fatty acid glucamides, alkylamidobetaines and/or protein fatty acid condensation products, the latter preferably being based on wheat proteins.

### Biogenic Active Ingredients

Biogenic active ingredients are to be understood as meaning, for example, tocopherol, tocopherol acetate, tocopherol palmitate, deoxyribonucleic acid, retinol, bisabolol, allantoin, phytantriol, AHA acids, amino acids, ceramides, pseudoceramides, essential oils, plant extracts and vitamin complexes.

### Antioxidants

Typical examples of such antioxidants are amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotinoids, carotenes, lycopene and derivatives thereof, chlorogenic acid and derivatives thereof, lipoic acid and derivatives thereof (e.g. dihydrolipoic acid), aurothioglycose, propylthiouracil and other thiols (e.g. thioredoxin, glutathione, cysteine, cystine, cystamine and the glycosyl, N-acetyl, methyl, ethyl, propyl, amyl, butyl, lauryl, palmitoyl, oleyl, linoleyl, cholesteryl and glyceryl esters thereof) and also salts thereof, dilauryl thiodipropionate, distearyl thiodipropionate, thiodipropionic acid and derivatives thereof (esters, ethers, peptides, lipids, nucleotides, nucleosides and salts) and also sulfoximine compounds (e.g. buthionine sulfoximines, homocysteine sulfoximine, buthionine sulfones, penta-, hexa-, hepta-thionine sulfoximine), also (metal) chelating agents (e.g. hydroxy fatty acids, palmitic acid phytic acid, lactoferrin), hydroxy acids (e.g. citric acid, lactic acid, malic acid), humic acid, bile acid, bile extracts, bilirubin, biliverdin, EDTA, EDDS, EGTA and derivatives thereof, unsaturated fatty acids and derivatives thereof (e.g. linolenic acid, linoleic acid, oleic acid), folic acid and derivatives thereof, ubiquinone and ubiquinol and derivatives thereof, vitamin C and derivatives (e.g. ascorbyl palmitate, magnesium ascorbyl phosphate, ascorbyl acetate), tocopherols and derivatives (e.g. vitamin E acetate), vitamin A and derivatives (e.g. vitamin A palmitate) and also coniferyl benzoate of benzoin resin, rutinic acid and derivatives thereof, glycosylrutin, ferulic acid, furfurylidene glucitol, carnosine, butyl hydroxytoluene, butyl hydroxyanisole, nordihydroguaiaretic acid, trihydroxybutyrophenone, uric acid and derivatives thereof, mannose and derivatives thereof, superoxide dismutase, N-[3-(3,5-di-tert-butyl-4-hydroxyphenyl) propionyl]sulfanilic acid (and salts thereof, for example the disodium salts), selenium and derivatives thereof (e.g. selenium methionine), stilbene and derivatives thereof (e.g. stilbene oxide, trans-stilbene oxide) and the derivatives suitable according to the invention (salts, esters, ethers, sugars, nucleotides, nucleosides, peptides and lipids) of those mentioned active ingredients. HALS (=“Hindered Amine Light Stabilizers”) compounds may also be mentioned.

### Preservatives and Bacteria-Inhibiting Agents

Suitable preservatives include, for example, Methyl-, Ethyl-, Propyl-, Butyl-parabens, Benzalkonium chloride, 2-Bromo-2-nitro-propane-1,3-diol, Dehydroacetic acid, Diazolidinyl Urea, 2-Dichloro-benzyl alcohol, DMDM hydantoin, Formaldehyde solution, Methyldibromoglutanitrile, Phenoxyethanol, Sodium Hydroxymethylglycinate, Imidazolidinyl Urea, Triclosan and further substance classes listed in the following reference: K. F. DePolo-A short textbook of cosmetology, Chapter 7, Table 7-2, 7-3, 7-4 and 7-5, p 210-219.

In certain embodiments, the formulation may include at least one moisturizer; at least one antioxidant; at least one emollient; and at least one antibacterial agent.

In some embodiments, the at least one moisturizer may be included in an amount of about 0.01 wt % to about 5 wt %, about 0.05 wt % to about 4.5 wt %, about 0.1 wt % to about 4 wt %, about 0.2 wt % to about 3.5 wt %, about 0.5 wt % to about 3 wt %, or about 1 wt % to about 2.5 wt %, based on total weight of the formulation.

In some embodiments, the at least one antioxidant may be included in an amount of about 0.5 wt % to about 5 wt %, about 1 wt % to about 4.5 wt %, about 1.5 wt % to about 4 wt %, about 2 wt % to about 3.5 wt %, or about 2.5 wt % to about 3 wt %, based on total weight of the formulation.

In some embodiments, the at least one emollient may be included in an amount of about 1 wt % to about 15 wt %, about 2 wt % to about 12 wt %, or about 5 wt % to about 10 wt %, based on total weight of the formulation.

In some embodiments, the at least one antibacterial agent may be included in an amount of about 1 wt % to about 10 wt %, about 2 wt % to about 9 wt %, about 3 wt % to about 8 wt %, or about 4 wt % to about 6 wt %, based on total weight of the formulation.

In some embodiments, the humectant may be included in an amount of about 0.1 wt % to about 5 wt %, about 0.5 wt % to about 4.5 wt %, about 1 wt % to about 4 wt %, about 1.5 wt % to about 3.5 wt %, or about 2 wt % to about 3 wt %, based on total weight of the formulation.

In some embodiments, the surfactant may be included in an amount of about 10 wt % about 40 wt %, about 15 wt % to about 35 wt %, or about 20 wt % to about 30 wt %, based on total weight of the formulation.

In some embodiments, the at least one cosmetically acceptable excipient may be included in an amount of about 0.001 wt % to about 25 wt %, about 0.005 wt % to about 22 wt %, about 0.01 wt % to about 20 wt %, about 0.05 wt % to about 18 wt %, about 0.1 wt % to about 15 wt %, about 0.5 wt % to about 12 wt %, about 1 wt % to about 10 wt %, about 2 wt % to about 8 wt %, or about 3 wt % to about 5 wt %, based on total weight of the topical formulation.

In another embodiment of the formulation, the formulation for conditioning may include glycerin, aloe vera, panthenol, water, honey, behentrimonium methosulfate (BTMS), argan oil, sunflower oil, carrot oil, sweet almond oil, castor oil, babassu oil, rosemary oleoresin extract, vitamin E, tea tree essential oil, cypress essential oil, peppermint essential oil, rosemary essential oil, radish root ferment, and Cocos nucifera fruit with Lactobacillus

In some embodiments, the conditioner formulation may include olive oil, such as extra virgin olive oil, to smooth scalp, provide moisture, elasticity and shine hair. In some embodiments, the sesame oil may include amino acids and protein, which fight psoriasis and eczema. In some embodiments, coconut oil may be included to soften the hair.

In some embodiments, the formulation may be formulated as a conditioner.

In some embodiments, a method of applying the formulations are provided. The methods including applying the formulations described herein to the scalp and/or hair.

A method of preparing the formulation for conditioning is also provided. The method includes combining a humectant, natural gum, and at least one moisturizer and mixing until smooth at a temperature of about 100 to about 150° C. Once smooth, adding water to the mixture and mixing the same with a hand mixer. The method may further include combining the remaining oils and BTMS with the mixture by heating at a temperature of about 125 to about 150° C. until melted. Once melted, the remaining ingredients are added, which are then mixed at a high speed until a homogenous mixture is formed.

### ILLUSTRATIVE EXAMPLES

The following examples are set forth to assist in understanding the invention and should not, of course, be construed as specifically limiting the invention described and claimed herein. Such variations of the invention, including the substitution of all equivalents now known or later developed, which would be within the purview of those skilled in the art, and changes in formulation or minor changes in experimental design, are to be considered to fall within the scope of the invention incorporated herein.

### Example 1

A formulation in accordance with embodiments described herein was formulated into a shampoo described in Table 1 below. The formulation of Example 1 may be applied to the skin.

TABLE 1
Shampoo Formulation

| Ingredient | Amount (wt. %) |
|---|---|
| Glycerin | 0.1 wt. %-10 wt. % |
| Xanthan Gum | 0.1 wt %-10 wt % |
| Water | 45 wt % to 80 wt % |
| Panthenol | 0.01 wt % to 5 wt % |
| Aloe Vera | 0.01 wt % to about 2 wt % |
| Honey | 0.01 wt % to 1 wt % |
| Argan Oil | 0.01 wt % to 1 wt % |
| Sunflower Oil | 0.01 wt % to 1 wt % |
| Carrot Oil | 0.01 wt % to 1 wt % |
| Sweet Almond Oil | 0.01 wt % to 1 wt % |
| Castor Oil | 0.01 wt % to 1 wt % |
| Rosemary Oleoresin Extract (ROE) | 0.01 wt % to 1 wt % |
| Vitamin E | 0.01 wt % to 1 wt % |
| Tea Tree Essential Oil | 0.1 wt % to 2 wt % |
| Cypress Essential Oil | 0.1 wt % to 2 wt % |
| Peppermint Essential Oil | 0.5 wt % to 5 wt % |
| Rosemary Essential Oil | 0.5 wt % to 5 wt % |
| Leucidal Liquid (radish root ferment) | 1 wt % to 10 wt % |
| AMTicide coconut (Cocos nucifera fruit with | 1 wt % to 10 wt % |
| Lactobacillus) | |
| Coco-Glucoside (glucose and coconut oil) | 10 wt % to 30 wt % |

The formulation of Example 1 was prepared by combining glycerin, xanthan gum, aloe vera powder, panthenol, and honey in a dish and were mixed until smooth. Once smoothed, water was added and mixed using a hand mixer at room temperature. The mixture was set aside. A second mixture was prepared by combining all oils and preservatives in a dish by mixing until smooth. The two mixtures were then combined, where the Coco-Glucoside was then slowly mixed in.

### Example 2

A formulation in accordance with embodiments described herein was formulated into a conditioner having the composition described in Table 2 below.

TABLE 2
Conditioner Formulation

| Ingredient | Amount (wt. %) |
|---|---|
| Glycerin | 1 wt % to 10 wt % |
| Aloe Vera | 0.1 wt % to 5 wt % |
| Panthenol | 0.01 wt % to 5 wt % |
| Water | 60 wt % to 90 wt % |
| Honey | 0.01 wt % to 2 wt % |
| Behetrimonium methosulfate (BTMS) | 1 wt % to 15 wt % |
| Argan Oil | 0.01 wt % to 1 wt % |
| Sunflower Oil | 0.01 wt % to 1 wt % |
| Carrot Oil | 0.01 wt % to 1 wt % |
| Sweet Almond Oil | 0.01 wt % to 1 wt % |
| Castor Oil | 0.01 wt % to 1 wt % |
| Babassu Oil | 0.01 wt % to 1 wt % |
| Rosemary oleoresin extract (ROE) | 0.01 wt % to 2 wt % |
| Vitamin E | 0.01 wt % to 2 wt % |
| Tea Tree Essential Oil | 0.1 wt % to 5 wt % |
| Cypress Essential Oil | 0.1 wt % to 5 wt % |
| Peppermint Essential Oil | 0.5 wt % to 5 wt % |
| Rosemary Essential Oil | 0.5 wt % to 5 wt % |
| Leucidal Liquid (radish root ferment) | 1 wt % to 10 wt % |
| AMTicide coconut (Cocos nucifera fruit with | 1 wt % to 10 wt % |
| Lactobacillus) | |

The formulation of Example 2 was prepared by combining glycerin, aloe vera, panthenol, and honey. Once smooth, water was added to the mixture which was heated to a temperature of 140° C. In a separate mixture, all the oils were then combined with BTMS and heated until melted at a temperature of about 140° C. A third mixture was then prepared by combining Vitamin E, ROE, essential oils, and preservatives at room temperature. The first two mixtures were combined at a temperature of about 140° C. using a high speed mixture. The mixture was then allowed to cool to about 100° C., where the third mixture was then slowly added using a high speed mixture. This mixture was mixed until uniform to form the final formulation.

For simplicity of explanation, the embodiments of the methods of this disclosure are depicted and described as a series of acts. However, acts in accordance with this disclosure can occur in various orders and/or concurrently, and with other acts not presented and described herein. Furthermore, not all illustrated acts may be required to implement the methods in accordance with the disclosed subject matter. In addition, those skilled in the art will understand and appreciate that the methods could alternatively be represented as a series of interrelated states via a state diagram or events.

In the foregoing description, numerous specific details are set forth, such as specific materials, dimensions, processes parameters, etc., to provide a thorough understanding of the present invention. The particular features, structures, materials, or characteristics may be combined in any suitable manner in one or more embodiments. The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Reference throughout this specification to “an embodiment”, “certain embodiments”, or “one embodiment” means that a particular feature, structure, or characteristic described in connection with the embodiment is included in at least one embodiment. Thus, the appearances of the phrase “an embodiment”, “certain embodiments”, or “one embodiment” in various places throughout this specification are not necessarily all referring to the same embodiment.

Reference throughout this specification to numerical ranges should not be construed as limiting and should be understood as encompassing the outer limits of the range as well as each number and/or narrower range within the enumerated numerical range.

The term “about”, when referring to a physical quantity, is to be understood to include measurement errors within, and inclusive of 10%. For example, “about 100° C.” should be understood to mean “100±10° C.”.

The present invention has been described with reference to specific exemplary embodiments thereof. The specification and drawings are, accordingly, to be regarded in an illustrative rather than a restrictive sense. Various modifications of the invention in addition to those shown and described herein will become apparent to those skilled in the art and are intended to fall within the scope of the appended claims.

1. A formulation comprising:
at least one moisturizer comprising argan oil or aloe vera;
at least one antioxidant comprising chamomile;
at least one preservative; and
at least one antibacterial agent comprising camphor.

2. The formulation of claim 1, wherein the at least one moisturizer further comprises sweet almond oil, castor oil, or peppermint essential oil.

3. The formulation of claim 1, wherein the at least one preservative comprises rosemary oleoresin, radish root ferment and Cocos nucifera fruit with Lactobacillus.

4. The formulation of claim 1, wherein the at least one antibacterial agent further comprises carrot oil, cypress essential oil, or tea tree essential oil.

5. The formulation of claim 1, further comprising a humectant.

6. The formulation of claim 5, wherein the humectant comprises panthenol, honey glycerin, or a combination thereof.

7. The formulation of claim 1, further comprising a surfactant.

8. The formulation of claim 7, wherein the surfactant is a reactant of glucose and coconut oil.

9. The formulation of claim 1, further comprising a botanical agent.

10. The formulation of claim 9, wherein the botanical agent comprises rosemary essential oil, ylang ylang, hemp seed oil, lavender, eucalyptus, or a combination thereof.

11. The formulation of claim 1, further comprising an emollient.

12. The formulation of claim 11, wherein the emollient comprises shea butter, cocoa butter, mineral oil, lanolin, petrolatum, paraffin, beeswax, squalene, cetyl alcohol, stearyl alcohol, olive oil, triethylhexanoin, coconut oil, jojoba oil, sesame oil, almond oil, or combinations thereof.

13.-15. (canceled)

16. A formulation comprising:
at least one moisturizer comprising aloe vera;
at least one emollient comprising coconut oil and sesame oil;
at least one antibacterial agent comprising tea tree essential oil; and
at least one antioxidant comprising Vitamin E.

17. The formulation of claim 16, wherein the at least one moisturizer further comprises babassu oil, argan oil, sweet almond oil, castor oil, or peppermint essential oil.

18. The formulation of claim 16, wherein the at least one antibacterial agent further comprises carrot oil, or cypress essential oil.

19. The formulation of claim 16, wherein the at least one antioxidant further comprises dandelion extract

20. The formulation of any one of claims 16-19, further comprising a humectant.

21. (canceled)

22. The formulation of claim 16, further comprising a surfactant.

23. (canceled)

24. The formulation of claim 16, further comprising a botanical agent.

25.-29. (canceled)

30. The formulation of claim 16, further comprising a quaternary ammonium salt.

31. (canceled)
